(19) United States
(12) Patent Application Publication
(10) Pub. No.: US 2007/0105793 A1
(43) Pub. Date: May 10, 2007

(54) Compositions and methods using nicotinic acid for treatment of hypercholesterolemia, hyperlipidemia nd cardiovascular disease

(76) Inventor: Curt Hendrix, Encino, CA (US)

Correspondence Address: KOPPEL, PATRICK & HEYBL, 555 ST. CHARLES DRIVE, SUITE 107, THOUSAND OAKS, CA, 91360, US

(21) Appl. No.: 11/591,164

(22) Filed: Oct. 31, 2006

Related U.S. Application Data
(60) Provisional application No. 60/733,406, filed on Nov. 4, 2005.
(60) Provisional application No. 60/742,115, filed on Dec. 2, 2005.
(60) Provisional application No. 60/758,037, filed on Jan. 10, 2006.

(51) Int. Cl. A61K 31/70; A01N 43/04

(52) U.S. Cl. 514/43

(57) ABSTRACT

An improved method and composition for the treatment of hyperlipidemia, hypercholesterolemia, and hypertriglyceridemia as well as other disease states, particularly cardiovascular problems, uses a multiple release system that provides an immediate release of nicotinic acid followed by a subsequent release of nicotinic acid from the hydrolysis of a compound that generates nicotinic acid. In general, a composition according to the present invention comprises: (1) a quantity of nicotinic acid intended to saturate liver enzymes during a period from about 0.5 hours to about 2.5 hours after administration of the pharmaceutical composition but insufficient to trigger significant generation of nicotinuric acid; (2) a quantity of a derivative or analogue of nicotinic acid that is subject to hydrolysis after ingestion such that preferrably no more than about 10% of the derivative or analogue is hydrolyzed by about 8 hours after ingestion and such that substantially all of the derivative or analogue is hydrolyzed by about 12 hours after ingestion; and (3) at least one pharmaceutically acceptable carrier.

Benefit is claimed of Provisional Applications Ser. No. 60/733,406 filed Nov. 4, 2005, Ser. No. 60/742,115 filed Dec. 2, 2005, and Ser. No. 60/758,037 filed Jan. 10, 2006.

### BACKGROUND OF THE INVENTION

This invention is directed to compositions and methods containing nicotinic acid (niacin) and chemical varients, precursors, derivatives or analogues thereof for the treatment of a broad range of diseases including, but not limited to, hypercholesterolemia, hyperlipidemia and cardiovascular disease.

Hypercholesterolemia, hyperlipidemia and cardiovascular disease are increasingly prevalent in Western industrial societies. The reasons for this are not completely understood, but may relate partly to a genetic predisposition to these conditions and partly to a diet high in saturated fats, together with an increasingly sedentary lifestyle as manual labor becomes increasingly less necessary. Hypercholesterolemia and hyperlipidemia are very significant, because they predispose individuals to cardiovascular disease, including atherosclerosis, myocardial infarction (heart attack), and stroke.

Specific forms of hyperlipidemia include, for example, hypercholesterolemia, familial dysbetalipoproteinemia, diabetic dyslipidemia, nephrotic dyslipidemia and familial combined hyperlipidemia. Hypercholesterolemia is characterized by an elevation in serum low density lipoprotein-cholesterol and serum total cholesterol. Low density lipoprotein (LDLcholesterol) transports cholesterol in the blood. Familial dysbetalipoproteinemia, also known as Type III hyperlipidemia, is characterized by an accumulation of very low density lipoprotein-cholesterol (VLDL-cholesterol) particles called betaVLDLs in the serum. Also associated with this condition is a replacement of normal apolipoprotein E3 with abnormal isoform apolipoprotein E2. Diabetic dyslipidemia is characterized by multiple lipoprotein abnormalities, such as an overproduction of VLDL-cholesterol, abnormal VLDL triglyceride lipolysis, reduced LDL-cholesterol receptor activity and, on occasion, Type III hyperlipidemia. Nephrotic dyslipidemia, associated with malfunction of the kidneys, is difficult to treat and frequently includes hypercholesterolemia and hypertriglyceridemia. Familial combined hyperlipidemia is characterized by multiple phenotypes of hyperlipidemia, i.e., Type IIa, IIb, IV, V or hyperapobetalipoproteinemia.

It is well known that the likelihood of cardiovascular disease can be decreased if the serum lipids, and in particular LDL-cholesterol, can be reduced. It is also well known that the progression of atherosclerosis can be retarded or the regression of atherosclerosis can be induced if serum lipids can be lowered. In such cases, individuals diagnosed with hyperlipidemia or hypercholesterolemia should consider lipid-lowering therapy to retard the progression or induce the regression of atherosclerosis for purposes of reducing their risk—of cardiovascular disease, and in particular coronary artery disease. Such therapy will reduce the risk of stroke and mycardial infarction, among other consequences. In addition, certain individuals with what are considered normal blood lipid levels can develop cardiovascular disease. In these individuals other factors like lipid peroxidation and high levels of Lp(a) can lead to atherogenesis despite relatively normal cholesterol and lipid levels.

Hypertriglyceridemia is also an independent risk factor for cardiovascular disease, such as coronary artery disease. Many people with hyperlipidemia or hypercholesterolemia also have elevated triglyceride levels. It is known that a reduction in elevated riglycerides can result in the secondary lowering of cholesterol. These individuals should also consider lipid-lowering therapy to reduce their elevated triglycerides for purposes of decreasing their incidence of atherosclerosis and coronary artery disease. Such therapy is also recommended for individuals who have already experienced an occurrence of stroke or myocardial infarction.

Cholesterol is transported in the blood by lipoprotein complexes, such as VLDL-cholesterol, LDL-cholesterol, and high density lipoproteincholesterol (HDL-cholesterol). LDL carries cholesterol in the blood to the subendothelial spaces of blood vessel walls. It is believed that peroxidation of LDL-cholesterol within the subendothelial space of blood vessel walls leads to atherosclerotic plaque formation. HDL-cholesterol, on the other hand, is believed to counter plaque formation and delay or prevent the onset of cardiovascular disease and atherosclerotic symptoms. Several subtypes of HDL-cholesterol, such as HDL₁-cholesterol, HDL₂-cholesterol and HDL₃- cholesterol, have been identified to date.

In the past, there have been numerous methods proposed for reducing elevated cholesterol levels and for increasing HDL-cholesterol levels. Typically, these methods include diet and/or daily administration of lipid-altering or hypolipidemic agents. Another method-proposed is based on periodic plasma delipidation by a continuous flow filtration system, as described in U.S. Pat. No. 4,895,558.

Several types of hypolipidemic agents have been developed to treat hyperlipidemia or hypercholesterolemia or normolipidemics diagnosed with cardiovascular disease. In general, these agents act (1) by reducing the production of the serum lipoproteins or lipids, or (2) by enhancing their removal from the serum or plasma. Drugs that lower the concentration of serum lipoproteins or lipids include inhibitors of HMG-CoA reductase, the rate controlling enzyme in the biosynthetic pathway of cholesterol. Examples of HMG-CoA reductase inhibitors include: (1) mevastatin, U.S. Pat. No. 3,983,140; (2) lovastatin, also referred to as mevinolin, U.S. Pat. No. 4,231,938; (3) pravastatin, U.S. Pat. Nos. 4,346,227 and 4,410,629; (4) lactones of pravastatin, U.S. Pat. No. 4,448,979; (5) velostatin, also referred to as synvinolin; (6) simvastatin, U.S. Pat. Nos. 4,448,784 and 4,450,171; (7) rivastatin; (8) fluvastatin; (9) atorvastatin; and (10) cerivastatin. For other examples of HMGCoA reductase inhibitors, see U.S. Pat. Nos. 5,217,992; 5,196,440; 5,189,180; 5,166,364; 5,157,134; 5,110,940; 5,106,992; 5,099,035; 5,081,136; 5,049,696; 5,049,577; 5,025,017; 5,011,947; 5,010,105; 4,970,221; 4,940,800; 4,866,058; 4,686,237; 4,647,576; European Application Nos. 0142146A2 and 0221025A1; and PCT Application Nos. WO 86/03488 and WO 86107054.

Other drugs which lower serum cholesterol include, for example, nicotinic acid, bile acid sequestrants, e.g., cholestyramine, colestipol DEA-Sephadex (Secholex® and Polidexide®), probucol and related compounds as disclosed in U.S. Pat. No. 3,674,836, lipostabil (Rhone-Poulenc), Eisai E5050 (an N-substituted ethanolamine derivative), imanixil (HOE-402) tetrahydrolipstatin (THL), isitigmastanyiphosphorylcholine (SPC, Roche), aminocyclodextrin (Tanabe Seiyoku), Ajirlomoto AJ-814 (azulene derivative), melinamide (Sumitomo), Sandoz 58-035, American Cyanamid CL-277,082 and CL-283,546 (disubstituted urea derivatives), ronicol (which has an alcohol which corresponds to nicotinic acid), neomycin, p-aminosalicylic acid, aspirin, quaternary amine poly(diallyldimethylammonium chloride) and ionenes such as disclosed in U.S. Pat. No 4,027,009, poly(diallylmethylamine) derivatives such as disclosed in U.S. Pat. No. 4,759,923, omega-3-fatty acids found in various fish oil supplements, fibric acid derivatives, e.g., gemfibrozil, clofibrate, bezafibrate, fenofibrate, ciprofibrate and clinofibrate, and other known serum cholesterol lowering agents such as those described in U.S. Pat. No. 5,200,424; European Patent Application No. 0065835A1, European Patent No. 164-698-A, G.B. Patent No. 1,586,152 and G.B. Patent Application No. 2162-179-A.

Nicotinic acid, also known as niacin, has been used for many years in the treatment of hyperlipidemia or hypercholesterolemia. This compound has long been known to exhibit the beneficial effects of reducing total cholesterol, VLDL-cholesterol and VLDL-cholesterol remnants, LDL-cholesterol triglycerides and apolipoprotein a, known as “Lp(a),” in the human body, while increasing desirable HDL-cholesterol.

Nicotinic acid has normally been administered three times per day after meals. This dosing regimen is known to provide a very beneficial effect on blood lipids as discussed in Knopp et al., “Contrasting Effects of Unmodified and Time-Release Forms of Niacin on Lipoproteins in Hyperlipidemic Subjects: Clues to Mechanism of Action of Niacin”; Metabolism (34)7:642-647 (1985). The chief advantage of this profile is the ability of nicotinic acid to decrease total cholesterol, LDL-cholesterol, triglycerides and Lp(a) while increasing HDLcholesterol particles. While such a regimen does produce beneficial effects, cutaneous flushing and the like still often occur in the hyperlipidemics to whom the nicotinic acid is administered.

In order to avoid or reduce the cutaneous flushing resulting from nicotinic acid therapy, a number of agents have been suggested for administration with an effective antihyperlipidemic amount of nicotinic acid, such as guar gum as reported in U.S. Pat. No. 4,965,252, mineral salts as disclosed in U.S. Pat. No. 5,023,245, inorganic magnesium salts as reported in U.S. Pat. No. 4,911,917, and non-steroidal anti-inflammatories, such as aspirin, as disclosed in PCT Application No. 96/32942. These agents have been reported to avoid or reduce the cutaneous flushing side effect commonly associated with nicotinic acid divided dose treatment.

Another method of avoiding or reducing the side effects associated with immediate release niacin is the use of extended or sustained release formulations. Extended or sustained release formulations are designed to slowly release the active ingredient from the tablet or capsule, which allows a reduction in dosing frequency as compared to the typical dosing frequency associated with conventional or immediate dosage forms. The slow drug release reduces and prolongs blood levels of the drug and, thus, minimizes or lessens the cutaneous flushing side effects that are associated with conventional or immediate release niacin products. Sustained release formulations of niacin have been developed, such as Nicobid® capsules (Rhone-Poulenc Rorer), Endur-acin® (Innovite Corporation), and the formulations described in U.S. Pat. Nos. 5,126,145 and 5,268,181, which describe a sustained release niacin formulation containing two different types of hydroxypropylmethylcelluloses and a hydrophobic component.

Studies in hyperlipidemic patients have been conducted with a number of sustained release niacin products. These studies have demonstrated that the sustained release products do not have the same advantageous lipid-altering effects as immediate release niacin, and in fact have a worse side effect profile compared to the immediate release product. The major disadvantage of the sustained release formulations, as reported in Knopp et al.: Metabolism, 34(7):642-647 (1985), is the significantly lower reduction in triglycerides (−2% for the sustained release versus −38% for the immediate release) and lower increase in HDL-cholesterol (+8% for the sustained release versus +22% for the immediate release) and HDL2-cholesterol particles, which are known by the art to be most beneficial (−5% for thie sustained release versus +37% for the immediate release).

Additionally, sustained release niacin formulations are known to cause greater incidences of liver toxicity, as described in Henken et al., Am J Med, 91:1991 (1991) and Dalton et al., Am J Med, 93: 102 (1992). There is also great concern regarding the potential of these formulations in disrupting glucose metabolism and uric acid levels.

In a previous edition of the Journal of the American Medical Association (JAMA), an article appeared which presented research results investigating the liver toxicity problems associated with a sustained release form of nicotinic acid. “A Comparison of the Efficacy and Toxic Effects of Sustained-vs. Immediate-Release Niacin in Hypercholesterolemic Patients”, McKenney et al., JAMA, 271(9): 672 (Mar. 2, 1994). The article presented a study of twenty-three patients. Eighteen patients (78%) were forced to withdraw because liver function tests (LFTs) increased indicating potential liver damage. The conclusion of the authors of that article was that the sustained release form of niacin “should be restricted from use.”

A similar conclusion was reached in an article by representatives of the Food and Drug Administration and entitled “Hepatic Toxicity of Unmodified and Time-Release Preparations of Niacin”, Rader et al., Am J Med, 92:77 (January, 1992). Because of these studies and similar conclusions drawn by other health care professionals, the sustained release forms of niacin have experienced limited utilization.

HMG-CoA reductase inhibitors have also been used for many years to treat hyperlipidemia. These compounds are known to exhibit beneficial effects of reducing total cholesterol and LDL-cholesterol in the human body and elevating HDL-cholesterol levels in some individuals. Grundy S M, N Engl J Med, 319 (1):24-32, at 25-26 and 31 (Jul. 7, 1988). The conversion of HMG-CoA to mevalonate is an early step in the biosynthesis of cholesterol. Inhibition of HMGCoA reductase, which interferes with the production of mevalonate, is the basis by which the HMG-CoA reductase inhibitors exert their total cholesterol-lowering and LDL-cholesterol-lowering effects. Grundy S M, N Engl J Med, 319(1):24-32, at 25 and 26 (Jul. 7, 1988).

HMG-CoA reductase inhibitors are not without drawback, however. HMG-CoA reductase inhibitors are known to induce hepatotoxicity, myopathy and rhabdomyolysis, as reported in, for example, Garnett W R, Am J Cardiol, 78 (Suppl 6A) 20-25 (Sep. 26, 1996), “The Lovastatin Pravastatin Study Group:, Am J Cardiol, 71:810-815 (Apr. 1, 1993), Dujovne C A et al., Am J Med, 91 (Suppl 1 B):25S-30S (Jul. 31, 1991); and Mantell G M et al., Am J. Cardiol, 66:11 B-1 5B (Sep. 8, 1990). Statins do not significantly reduce triglycerides and result in minimal increase of HDL. Additionally they have little impact on Lp(a) and may even increase it.

Moreover, the Physicians' Desk Reference (PDR) 50th Ed., page 1700, column 3, 1996, reports that lovastatin, an HMG-CoA reductase inhibitor should be used with caution in patients who have a past history of liver disease, and that lovastatin therapy is contraindicated for those individuals with active liver disease or unexplained persistent elevations of serum transaminases. The 1996 PDR further reports (page 1701, column 1) that rhabdomyolysis has been associated with lovastatin therapy alone and when combined with lipid-lowering doses (˜1 g/day) of nicotinic acid, and that physicians contemplating combined therapy with lovastatin and lipid-lowering doses of nicotinic acid should carefully weigh the potential benefits and risks and should carefully monitor individuals for any signs and symptoms of muscle pain, tenderness, or weakness, particularly during the initial months of therapy and during any periods of upward dosage titration of either drug. The 1996 PDR further reports (page 1701, column 1) that cases of myopathy have been associated with patients taking lovastatin concomitantly with lipid-lowering doses of nicotinic acid. Similar contraindications (1) for fluvastatin on (PDR, page 2267—column 3, page 2268, column 1), (2) for pravastatin (PDR, page 767, column 1), and (3) for simvastatin (PDR, page 1777, column 2). Still further, the PDR recommends that concomitant therapy with HMG-CoA reductase inhibitors and lipid lowering doses of nicotinic acid is generally not recommended (PDR, page 768, column 3). Accordingly, these agents have the potential for causing serious side effects, particularly in individuals who have liver problems or other problems that can predispose them to these side effects.

Notwithstanding the recommendations in the 1996 PDR, Grundy S M, N Engl. J Med, 319 (1):24-33 (Jul. 7, 1988), reports that HMG-CoA reductase inhibitors when used alone (pages 29-30) and nicotinic acid when used alone (page 24) are effective in reducing elevated cholesterol plasma levels. Grundy further reports that “[b]ecause of their efficacy . . . bile acid sequestrants (cholestyramine and colestipol) and niacin are probably the drugs of first choice for hypercholesterolemia. Although these drugs can be highly effective and are satisfactory for use in many patients with high cholesterol levels, they unfortunately are not well tolerated by all patients. Therefore, in spite of their proved usefulness, bile acid sequestrants and niacin are not ideal cholesterol-lowering agents” (page 24, column 2, lines 10-25). Still further, Grundy reports that the “. . . administration of [HMG-CoA] reductase inhibitors twice a day is somewhat more effective than administration once a day, at the same total dosage” (page 30, column 1, lines 13-17). Grundy also reports “. . . that the combination of lovastatin and cyclosporine, gemfibrozil or nicotinic acid may predispose patients to myopathy and occasionally even to rhabdomyolysis” (page 29, column 1, lines 7-11). Still further, that “[the combination of lovastatin and niacin has not been shown to be safe in a controlled clinical trial; furthermore, a manifestation of an adverse interaction between the agents, such as myopathy, could occur” (Grundy, page 30, column 1, lines 54-59). Gardner S F et al., Pharmacotherapy, 16 (3):421-423 (1996); Pastemak R C et al., Ann Intern Med, 125 (7):529-540 (Oct. 1, 1996), O'Keefe J H et al., Am J Cardiol, 76:480-484 (Sep. 1, 1995), and Davignon J et al., Am J Cardiol, 73:339345 (Feb. 15, 1994) also address these issues.

In Vacek J L et al., Am J Cardiol, 76:182-184 (Jul. 15, 1995), it is reported that “. . . because of the present state of knowledge of the risks of hepatotoxicity with slow-release forms of nicotinic acid, this form of the drug should probably not be used [in combination with lovastatin] in future trials or clinical practice.”

Consistent with the reports by Vacek J L et al. and the 1996 PDR, the article by Jacobson T A and Amorosa L F, Am J Cardiol, 73:25D-29D (May 26, 1994), reports, on pages 28D-29D, that because “[a]bnormalities in liver enzyme profiles and fulminant hepatic failure have also been associated with the use of niacin, particularly sustained-release preparations . . . the use of fluvastatin in combination with a sustained release niacin preparation cannot generally be recommended based upon this study, which only examined crystalline or immediate release niacin.”

Current products on the market for delivery of niacin can be classified as immediate release, extended release or slow release forms. Immediate release compositions contain from about 25 mg to about 3,000 mg of niacin. The niacin reaches the blood stream in about 0.5 hours and is all released in about 2.5 hours. Extended release compositions, such as Niaspan™, contain from about 100 mg to about 3,000 mg of niacin. About 6-20% of this extended release product shows up in the blood stream 0.5-2.5 hours following ingestion with about 75% of the niacin showing up around 5-9 hours following ingestion with a Tₘₐₓ, of 5.6 to 6 hours (U.S. Pat. No. 6,818,229). Further, it is set forth in U.S. Pat. No. 6,080,428 that Niaspan™ is to be provided once per day in the evening or night (i.e., “once per day before going to bed”). Slow release forms contain about the same amount of niacin. However, the slow release products do not begin to show up in the blood stream until 10 hours following ingestion and continue to be released until about 24 hours from ingestion.

Immediate release and extended release forms have similar efficacy in reducing blood lipids; however, the extended release form delays that effect for several hours. The extended release form is advertised as resulting in less flushing than the immediate release form. Also, the slow release forms are less efficient at reducing blood lipids and have a tendency to increase liver enzymes. However, they have a reduced incidence of flushing when compared to the other two forms. The use of nicotinic acid and nicotinic acid derivatives to treat dysregulation of lipid metabolism has also been described in G. Domer & F. W. Fischer, “Zur Beeinflussung der Serumlipide and -lipoproteins durch den Hexanicotinsaureester des m-Inositol,” Arzneim. Forsch. 11: 110-113 (1961); A.M.A. El-Enein et al., “The Role of Nicotinic Acid and Inositol Hexanicotinate as Anticholesterolemic and Antilipemic Agents,” Nutrition Rep. Int'l 28: 899-911 (1983); V. Hutt et al., “Zur Wirkung einer Clofibrat-Inositolnicotinat-Kombination auf Lipide and Lipoproteine bei primarer Hyperlipoproteinamie der Typen IIa, IV and V,” Arzneim. Forsch. 33: 776-779 (1983); W. Kruse et al., “Nocturnal Inhibition of Lipolysis in Man by Nicotinic Acid and Derivatives,” Eur. J. Clin. Pharmacol. 16: 11-15 (1979); and J. G. Wechsler et al., “Lipids and Lipoproteins in Hyperlipidemia Type I1a During Treatment with Different Lipid Lowering Drugs,” Artery 8: 519-529 (1980). Studies have shown that phosphatidylinositol (PI) can stimulate reverse cholesterol transport by enhancing the flux of cholesterol into HDL and by promoting the transport of HDL-cholesterol to the liver and bile.

Therefore, there is a need for improved compositions and methods that can be used to treat hyperlipidemia, hypercholesterolemia and hypertriglyceridemia, or can be used to lower blood lipid levels, blood cholesterol levels, or blood triglyceride levels in patients with normal levels of these physiological parameters who are at risk of cardiovascular disease or who have already experienced an episode of cardiovascular disease. There is further a need for improved compositions and methods that improve other cardiovascular risk factors like lipid peroxidation, and levels of Lp(a) and avoid the side effects such as flushing associated with the administration of nicotinic acid and that also avoid the risks of liver and muscle damage associated with the statins and other anti-lipidemic drugs. There is further a need for improved compositions that reverse cardiovascular plaques. There is further a need for improved compositions that provide protection for an extended period of time without complex dosing regimens. Furthermore, there is a need for improved compositions and methods that are particularly beneficial to individuals at risk for cardiovascular disease because of existing diabetic symptoms or metabolic syndrome, and can be used to treat cardiovascular disease.

### SUMMARY OF THE INVENTION

One aspect of the invention is a composition for treatment of a dysregulation of lipid metabolism comprising:

(1) a quantity of nicotinic acid intended to be sufficient to saturate liver enzymes during a period from about 0.5 hours to about 2.5 hours after administration of the pharmaceutical composition but insufficient to trigger significant generation of nicotinuric acid;

(2) a quantity of a derivative or analogue of nicotinic acid that is subject to hydrolysis after ingestion such that preferably no more than about 10% of the derivative or analogue is hydrolyzed by about 8 hours after ingestion and such that substantially all of the derivative or analogue is hydrolyzed by about 12 hours after ingestion; and

(3) at least one pharmaceutically acceptable carrier.

In one alternative, the derivative or analogue of nicotinic acid that is subject to hydrolysis is an ester or amide of nicotinic acid. A particularly preferred ester or amide of nicotinic acid is inositol hexanicotinate (IHN). Another alternative is phospholipids of niacin or inositol such as phosphatidylinositol which may be added to niacin or the niacin plus derivative or analogue combination.

In another alternative, the ester or amide of nicotinic acid is selected from the group consisting of:

(1) nicotinyl alcohol tartrate and derivatives thereof;

(2) D-glucitol hexanicotinate and derivatives thereof;

(3) aluminum nicotinate and derivatives thereof;

(4) pentaerythrityl tetranicotinate (niceritrol) and derivatives thereof;

(5) D,L-α-tocopheryl nicotinate and derivatives thereof;

(6) nicotinamide;

(7) nicotinamide N-oxide;

(8) 6-hydroxynicotinamide;

(9) nicotinamide adenine dinucleotide (NAD);

(10) N-methyl-2-pyridine-8-carboxamide;

(11) N-methylnicotinamide;

(12) N-ribosyl-2-pyridone-5-carboxamide;

(13) N-methyl-4-pyridone-5-carboxamide;

(14) 1,3,3,6-tetranicotinyl-D-fructose (Bradilan™);

(15) an ester of ronicol (nicotinyl alcohol) selected from the group consisting of a lower alkyl ester, an aryl ester, an aralkyl ester, and an alkaryl ester; and

(16) an ester of nicotinic acid selected from the group consisting of a lower alkyl ester, an aryl ester, an aralkyl ester, and an alkaryl ester.

In still another alternative, the analogue or derivative of nicotinic acid is selected from the group consisting of;

(1) an ester or amide of 3-pyridinecarboxylic acid;

(2) an ester or amide of 3-pyridinecarbonitrile-1-oxide;

(3) a monoester, diester, monoamide, or diamide of 3,4pyridinedicarboxylic acid;

(4) an ester or amide of nipecotic acid;

(5) an ester or amide of 1-methyl-3-piperidinecarboxylic acid;

(6) an ester or amide of 3-carboxymethylpyridine;

(7) an ester or amide of 3-carboxyethylpyridine;

(8) an ester or amide of 3-carboxy-1-methylpyridinium choride;

(9) an ester or amide of 5-bromo-3-pyridinecarboxylic acid;

(10) an ester or amide of β-oxo-3-pyridinepropionic acid;

(11) an ester or amide of 2-acetyl-3-pyridinecarboxylic acid;

(12) a monoester, diester, monoamide, or diamide of [2,2′-dipyridine]-5,5′-dicarboxylic acid;

(13) an ester or amide of 2-chloro-3-pyridinecarboxylic acid;

(14) an ester or amide of 6-chloro-3-pyridinecarboxylic acid; and

(15) an ester or amide of 6-hydroxy-pyridinecarboxylic acid.

Typically, the composition is formulated for oral administration.

Typically, the pharmaceutical composition comprises from about 1 mg to about 2000 mg nicotinic acid per unit dose, For example, the pharmaceutical composition can-comprise nicotinic acid in a quantity per unit dose selected from the group consisting of 10 mg per unit dose, 15 mg per unit dose, 20 mg per unit dose, 25 mg per unit dose, 30 mg per unit dose, 35 mg per unit dose, 40 mg per unit dose, 50 mg per unit dose, 60 mg per unit dose, 70 mg per unit dose, 80 mg per unit dose, 90 mg per unit dose, 100 mg per unit dose, 150 mg per unit dose, 200 mg per unit dose, 250 mg per unit dose, 300 mg per unit dose, 350 mg per unit dose, 400 mg per unit dose, 450 mg per unit dose, 500 mg per unit dose, 600 mg per unit dose, 700 mg per unit dose, 800 mg per unit dose, 900 mg per unit dose, 1000 mg per unit dose, 1100 mg per unit dose, 1200 mg per unit dose, 1300 mg per unit dose, 1400 mg per unit dose, 1500 mg per unit dose, 1600 mg per unit dose, 1700 mg per unit dose, 1800 mg per unit dose, 1900 mg per unit dose, and 2000 mg per unit dose. If the pharmaceutical composition comprises IHN, typically the composition comprises from about 100 mg to about 3000 mg of IHN per unit dose. However, up to about 5 gms of IHN may be delivered.

The composition can further comprise pantethine and/or phospholipids of inositol in therapeutically effective quantities. If the composition comprises pantethine, typically the composition comprises from about 50 mg to about 3000 mg of pantethine per unit dose. If the composition comprises phosphatidylinositol the unit dose preferrably includes about 250 mg to about 6 grams of the inositol phospholipid.

The composition can further comprise at least one ingredient selected from the group consisting of policosanol, phytosterols, tocotrienols, calcium, statin drugs, fibrates, bile acid sequestrants, and guar gum in a therapeutically effective quantity. Alternatively, the composition can further comprise at least one ingredient selected from the group consisting of L-lysine, L-proline, vitamin C, vitamin E, and another antioxidant effective in preventing lipid peroxidation in a therapeutically effective quantity. In still another alternative, the composition can further comprise a matrix of inert vegetable waxes and/or cellulose fibers that slows the disintegration of the composition, in order that the level of the nicotinic acid in the blood due to the quantity of nicotinic acid sufficient to saturate liver enzymes in the composition is maximized at about 2 to 3 hours after ingestion instead of at about 45 minutes without the matrix. In this alternative, the composition can be in the form of a tablet.

Another aspect of the invention is a method of treating a dysregulation of lipid metabolism comprising the step of administering the composition according to the present invention as described above to a subject suffering from or at risk of suffering from a dysregulation of lipid metabolism in a quantity effective to treat the dysregulation of lipid metabolism.

The dysregulation of lipid metabolism can be hyperlipidemia, hypercholesterolemia, or hypertriglyceridemia. The subject can have experienced an episode of cardiovascular disease prior to administration of the composition. Alternatively, the subject can be at risk of an episode of cardiovascular disease. In still another alternative, the subject can be diabetic or have insulin resistance; compositions including nicotinic acid, inositol hexanicotinate (IHN), and pantethine are particularly useful for the treatment of subjects who are diabetic or who have insulin resistance.

Typically, the composition is administered once daily, in the mid- to late afternoon, preferrably with a snack or meal; however, administration can be performed at other times or with other frequencies or without food.

Yet another aspect of the invention is a method for the treatment of cardiovascular disease comprising the step of administering a composition according to the present invention to a subject suffering from or at risk for cardiovascular disease in a quantity effective to reverse accumulation of cardiovascular plaques.

### BRIEF DESCRIPTION OF THE DRAWINGS

The following invention will become better understood with reference to the specification, appended claims, and accompanying drawings, where:

FIG. 1 is a graph showing blood levels of nicotinic acid as a function of time after ingestion with prior available immediate-release nicotinic acid, extended release nicotinic acid and slow release nicotinic acid compared to compositions according to the present invention.

### DETAILED DESCRIPTION OF THE INVENTION

A new approach to the use of nicotinic acid (niacin) and related compounds for the treatment of conditions including, but not limited to, hyperlipidemia, hypercholesterolemia, and hypertriglyceridemia involves a multiple release system.

Niacin is subject to strong “first pass effect” in the liver. That means that the liver converts nicotinic-acid (niacin) to metabolites like niacinamide and NAD. These forms of niacin do not lower blood lipids (this is referred to as the second pathway). Another pathway (referred to as the first pathway) occurs when enough nicotinic acid is ingested so that the liver enzymes that are involved in converting nicotinic acid to niacinamide and/or NAD are “saturated” (used up), leading to systemic distribution of nicotinic acid. The nicotinic acid released into the bloodstream via the first pathway can then be converted to nicotinuric acid (NUA), which is the glycine conjugate of nicotinic acid. The second pathway results in the generation of a number of metabolites. These include nicotinamide (NAM), 6-hydroxynicotinamide (6HN), nicotinamide N-oxide (MNO), N-methylnicotinamide (MNA) and nicotinamide adenine dinucleotide (NAD). Additionally, MNA can be further metabolized into N-methyl 2-pyridone-5-carboxamide (2PY) and N-methyl-4-pyridone-5-carboxamide (4PY). It is the generation of the metabolites of the second pathway that are believed to be involved in the hepatotoxicity of nicotinic acid.

Compounds such as nicotinic acid that are subject to a first pass metabolism are considered to have non-linear pharmacokinetics. When a compound has non-linear pharmacokinetics, an increase or decrease in the dose administered will not necessarily produce the corresponding increase or decrease in observed blood levels. This is believed to be dependent on whether the metabolic level of the liver and its capacity to metabolize the compound have been exceeded. Therefore, it is generally accepted that the percent of administered nicotinic acid dose metabolized before the compound leaves the liver is dependent on the dose administered and the release rate.

The amount of nicotinic acid ingested and how fast it is released in the liver determines how quickly the enzymes are saturated and how much nicotinic and NUA get into systemic distribution. The greater the quantity of nicotinic acid that gets into the system (i.e., gets past the liver), the greater is the potential for flushing. Because only a small percentage of the extended release product is released in the first few hours after ingestion, it does not trigger as much of the first pathway metabolites (NUA) and therefore supposedly reduces flushing. In contrast, the slow release forms release the nicotinic acid so slowly that they very slowly saturate the enzymes, causing the liver to process the nicotinic acid over much longer periods of time, which potentiates elevated liver enzymes, and does not trigger as much NUA (pathway one) release into the system, therefore not reducing blood lipids to the same extent.

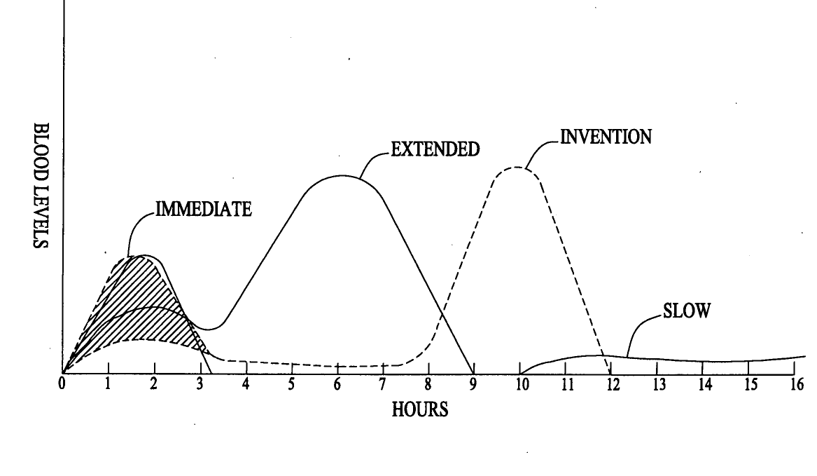

In order to provide an effective blood level of niacin and niacin metabolites to reduce serum lipids while minimizing flushing a multiple release system is described herein. It comprises a combination of enough immediate release nicotinic acid believed to be sufficient to saturate the enzymes within the first 0.5-2.5 hours but not enough excess to trigger any amount of NUA (pathway one) that would cause significant, if any, flushing (referred to as a “turn-off dosage”). Various amounts of niacin (as represented by the cross hatched area from 0 to 3 hours in FIG. 1) can be included in the formulation to obtain the turn-off dosage and control overflow into the blood system at levels which minimize flushing. To this immediate release dose of nicotinic acid a quantity of a derivative or analogue of nicotinic acid that is subject to hydrolysis after ingestion, such as inositol hexanicotinate (IHN), is added. IHN has been shown to have “zero” flush, no negative impact on blood sugar and no effect on liver enzymes. The desired lowering effect on serum lipids by IHN occurs as a result of release into the system of niacin by hydrolysis of IHN absorbed by the body or carried in the blood stream around 10 hours after ingestion. This is in contrast to presently available extended release compositions which control availability of niacin by using swelling agents that release the nicotinic acid in the gastrointestinal tract over a period of time. In a preferred combination about 15-35% by weight of the niacin ingested is provided by the immediate release niacin, which provides the “turn-off dosage,” and only small amounts of niacin reach the blood. It is believed that all or substantially all of the IHN reaches the blood stream. The remainder of the composition provided by IHN is absorbed rapidly following ingestion but is hydrolyzed in the body over an extended period of time. Because many, if not all of the enzymes that convert nicotinic acid to niacinamide and NAD are saturated by the immediate release of niacin, most if not all of the nicotinic acid released from the IHN will be released into the system and not converted to the non-lipid lowering metabolites (niacinamide/NAD). In other words, the enzyme saturation potentiates the effect of the nicotinic acid metabolites that are effective in lowering blood lipid levels by preventing diversion of the nicotinic acid into metabolites that are ineffective in lowering blood lipid levels. While the 15-35%_{w} niacin is preferred, the composition may include from about 1% to about 50%_{w} with the balance being IHN.

Accordingly, a composition for treatment of a dysregulation of lipid metabolism according to the present invention comprises:

(1) a quantity of nicotinic acid intended to be sufficient to saturate liver enzymes during a period from about 0.5 hours to about 2.5 hours after administration of the pharmaceutical composition but insufficient to trigger significant generation of nicotinuric acid;

(2) a quantity of a derivative or analogue of nicotinic acid that is subject to hydrolysis after ingestion such that preferrably no more than about 10% of the derivative or analogue is hydrolyzed by about 8 hours after ingestion and such that substantially all of the derivative or analogue is hydrolyzed by about 12 hours after ingestion; and

(3) at-least one pharmaceutically acceptable carrier.

Typically, the derivative or analogue of nicotinic acid that is subject to hydrolysis is an ester or amide of nicotinic acid. A particularly preferable ester of nicotinic acid that is useful in compositions and methods according to the present invention is inositol hexanicotinate (IHN). While the IHN may be a combination of the various natural or synthesized isomers of inositol, it is contemplated that the IHN may include one or more of the inositol isomers and the concentrations or ratios of these various isomers may be selected to vary or maximize the therapeutic effect of IHN. Synthetic isomers of inositol are the epi-cis- and allo-forms. Naturally occurring isomers of inositol are the myo-, scyllo-, muco-, neo-, D-chiro, and L-chiro forms. Refrence herein to IHN or inositol is also a reference to the isomers of inositol.

Other esters or amides of nicotinic acid or of derivatives or analogues of nicotinic acid that are useful in compositions and methods according to the present invention include, but are not limited to: nicotinyl alcohol tartrate and derivatives thereof; D-glucitol hexanicotinate and derivatives thereof; aluminum nicotinate and derivatives thereof; pentaerythrityl tetranicotinate (niceritrol) and derivatives thereof; D,L-α-tocopheryl nicotinate and derivatives thereof, nicotinamide; nicotinamide N-oxide; 6-hydroxynicotinamide; nicotinamide adenine dinucleotide (NAD); N-methyl-2pyridine-8-carboxamide; N-methylnicotinamide; N-ribosyl-2-pyridone-5carboxamide; N-methyl-4-pyridone-5-carboxamide; 1,3;3,6-tetranicotinyl-Dfructose (Bradilari); esters of ronicol (nicotinyl alcohol), such as lower alkyl esters, aryl esters, aralkyl esters, and alkaryl esters; and esters of nicotinic acid such as lower alkyl esters, aryl esters, aralkyl esters, and alkaryl esters. As used herein, the term “lower alkyl” refers to a branched or unbranched C₁-C₆ carbon moiety that can be noncyclic or cyclic and can be saturated or unsaturated; the moiety can be optionally substituted. Typically, the moiety is an unbranched, noncyclic, saturated, nonsubstituted moiety such as methyl, ethyl, propyl, butyl, pentyl, or hexyl.

In another alternative, the analogue or derivative of nicotinic acid can be selected from the group consisting of: (1) an ester or amide of 3pyridinecarboxylic acid; (2) an ester or amide of 3-pyridinecarbonitrile-1-oxide; (3) a monoester, diester, monoamide, or diamide of 3,4-pyridinedicarboxylic acid; (4) an ester or amide of nipecotic acid; (5) an ester or amide of 1-methyl-3-piperidinecarboxylic acid; (6) an ester or amide of 3-carboxymethyl pyridine; (7); an ester or amide of 3-carboxyethylpyridine; (8) an ester or amide of 3-carboxyl-methylpyridinium chloride; (9) an ester or amide of 5-bromo-3 pyridinecarboxylic acid; (10) an ester or amide of (3-oxo-3-pyridinepropionic acid; (11) an ester or amide of 2-acetyl-3-pyridinecarboxylic acid; (12) a monoester, diester, monoamide, or diamide of [2,2′-dipyridine]-5,5′-dicarboxylic acid; (13) an ester or amide of 2-chloro-3-pyridinecarboxylic acid; (14) an ester or amide of 6-chloro-3-pyridinecarboxylic acid; and (15) an ester or amide of 6-hydroxy pyridinecarboxylic acid. In this alternative, the ester is typically a lower alkyl ester, an aryl ester, an aralkyl ester, or an alkaryl ester. The amide is typically a lower alkylamide, an arylamide, an aralkyl amide, or an alkaryl amide.

Alternatively, other derivatives of nicotinic acid can be used that are subject to hydrolysis after ingestion such that no more than about 10% of the derivative or analogue is hydrolyzed by about 8 hours after ingestion and such that substantially all of the derivative or analogue is hydrolyzed by about 12 hours after ingestion.

Suitable pharmaceutical carriers are well known in the art. Because extended release or slow release of the nicotinic acid derivative or analogue is not required, it is not necessary to use coatings or systems that are required to provide extended release or slow release. Relatively simple formulations therefore can be employed.

In an additional embodiment, the composition further comprises a matrix of invert vegetable waxes and/or cellulose fibers that slows the disintegration of the composition, such as in the form of a tablet, in order that the level of the nicotinic acid in the blood due to the immediate release component of the composition, in other words, the quantity of nicotinic acid sufficient to saturate liver enzymes employed in the composition, is maximized at about 2 to 3 hours after ingestion instead of at about 45 minutes without the matrix. Suitable matrix are known in the art.

For example, but not by way of limitation, exemplary pharmaceutically acceptable carriers include, but are not limited to, any and/or all of solvents, including aqueous and non-aqueous solvents, dispersion media, matrices, antibacterial and/or antifungal agents, isotonic and/or absorption delaying agent, and/or the like, as well as pharmaceutical excipients such as, but not limited to, calcium carbonate, calcium phosphate, various sugars or types of starch, cellulose derivatives, gelatin, vegetable oils, polyethylene glycols and physiologically compatible solvents. The use of such media and/or agents for pharmaceutically active substances is well known in the art. Except insofar as any conventional medium, carrier, or agent is incompatible with the active ingredient or ingredients, its use in a composition according to the present invention is contemplated. Supplementary active ingredients can also be incorporated into the compositions, especially as described below under combination therapy. For administration of any of the compounds used in the present invention, preparations should meet sterility, pyrogenicity, general safety, and purity standards as required by the FDA Office of Biologics Standards or by other regulatory organizations regulating drugs.

Thus, the composition can be formulated for oral, buccal, sublingual, inhalation, insufflation, or parenteral administration. However, typically, oral administration is preferred.

If the composition is administered orally, it is typically administered in a conventional unit dosage form such as a tablet, a capsule, a pill, a troche, a wafer, a powder, or a liquid such as a solution, a suspension, a tincture, or a syrup. Oral formulations typically include such normally employed excipients as, for example, pharmaceutical grades of mannitol, lactose, starch, magnesium stearate, sodium saccharin, cellulose, magnesium carbonate, and other conventional pharmaceutical excipients. In certain defined embodiments, oral pharmaceutical compositions will comprise an inert diluent and/or assimilable edible carrier, and/or they may be enclosed in hard or soft shell gelatin capsules. Alternatively, they may be compressed into tablets. As another alternative, particularly for veterinary practice, they can be incorporated directly into food. For oral therapeutic administration, they can be incorporated with excipients or used in the form of ingestible tablets, buccal tablets, dragees, pills, troches, capsules, wafers, or other conventional dosage forms.

The tablets, pills, troches, capsules, wafers, or other conventional dosage forms can also contain for example: a binder, such as gum tragacanth, acacia, cornstarch, sorbitol, mucilage of starch, polyvinylpyrrolidone, or gelatin; excipients or fillers such as dicalcium phosphate, lactose, microcrystalline cellulose, or sugar; a disintegrating agent such as potato starch, croscarmellose sodium, or-sodium starch glycolate, or alginic acid; a lubricant such as magnesium stearate, stearic acid, talc, polyethylene glycol, or silica; a sweetening agent, such as sucrose, lactose, or saccharin; a wetting agent such as sodium lauryl sulfate; or a flavoring agent, such as peppermint, oil of wintergreen, orange flavoring, or cherry flavoring. When the dosage unit form is a capsule, it can contain, in addition to materials of the above types, a liquid carrier. Various other materials can be present as matrices or to otherwise modify the physical form and properties of the dosage unit. For instance, tablets, pills, or capsules can be glazed with shellac, sugar, or both. The compositions of the present invention may be manufactured in a manner that is itself known, e.g., by means of conventional mixing, dissolving, granulating, dragee-making, levitating, emulsifying, encapsulating, entrapping or lyophilizing processes.

Preparations for oral use can be obtained by combining the active compounds with solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added.

Suitable compositions comprise from about 1 mg to about 2000 mg nicotinic acid in a rapid release form per unit dose, in combination with preferrably from about 100 mg to about 3000 mg of IHN per unit dose or other derivative or analogue of nicotinic acid that is subject to hydrolysis after ingestion such that no more than about 10% of the derivative or analogue is hydrolyzed by about 8 hours after ingestion and such that substantially all of the derivative or analogue is hydrolyzed by about 12 hours after ingestion. Suitable quantities of nicotinic acid in a rapid release form include, but are not limited to, 10 mg per unit dose, 15 mg per unit dose, 20 mg per unit dose, 25 mg per unit dose, 30 mg per unit dose, 35 mg per unit dose, 40 mg per unit dose, 50 mg per unit dose, 60 mg per unit dose, 70 mg per unit dose, 80 mg per unit dose, 90 mg per unit dose, 100 mg per unit dose, 150 mg per unit dose, 200 mg per unit dose, 250 mg per unit dose, 300 mg per unit dose, 350 mg per unit dose, 400 mg per unit dose, 450 mg per unit dose, 500 mg per unit dose, 600 mg per unit dose, 700 mg per unit dose, 800 mg per unit dose, 900 mg per unit dose, 1000 mg per unit dose, 1100 mg per unit dose, 1200 mg per unit dose, 1300 mg per unit dose, 1400 mg per unit dose, 1500 mg per unit dose, 1600 mg per unit dose, 1700 mg per unit dose, 1800 mg per unit dose, 1900 mg per unit dose, or 2000 mg per unit dose, as well as other intermediate doses between 10 mg per unit dose and 2000 mg per unit dose. As an alternative, other compounds that can provide niacin or nicotinic acid to the blood stream once the liver enzymes have been shut off may be used to replace excess ingested niacin, which causes flushing. However, the compositions can include up to about 5 gm of IHN.

FIG. 1 is a graphical representation of the blood levels of niacin provided by the combination product described herein compared to equivalent amounts of niacin provided to the blood stream (i.e., not consumed in the liver) by slow release products presently available. Because the IHN appears to be absorbed from the intestinal tract intact but takes several hours to hydrolyze in the body, the combination product provides niacin to the blood starting approximately 8 hours after ingestion. Therefore it is recommended that the combination product be ingested by the patient during the mid- or late afternoon, preferrably between 4-6 pm with a snack or meal and not just prior to going to bed.

As a further embodiment, pantethine in a pharmaceutically effective quantity may be added to the niacin/IHN combination or combination of niacin with the derivative or analogue of nicotinic acid that is subject to hydrolysis after ingestion as described above. Pantethine, a derivative of Vitamin B₅, is the stable disulfide form of pantetheine, which is the chief precursor to Coenzyme A (CoA). Pantethine supports lipid metabolism by its ability to raise levels of CoA, a cofactor involved in several metabolic pathways including carbohydrate and lipid metabolism, and may work like an HMG CoA reductase inhibitor. HMG-CoA reductase inhibitors are a group of prescription drugs, commonly referred to as statin drugs, used to lower cholesterol. These medicines work by slowing down the body's ability to make cholesterol. Statins, such as lovastatin, have been shown to be synergistic when combined with niacin (Advicor®, an extended release Niacin/lovastatin combination available from KOS Pharmaceutical); however, as described above, the combination of statins and niacin can pose severe risks. The combination of niacin and IHN disclosed herein, with the further addition of pantethine, can be particularly beneficial in diabetics without the risks of statin use.

Cardiovascular disease risk is elevated in both Type I and Type II diabetics. Pantethine lowers β-thromboglobulin. On the other hand, niacin can raise blood sugar levels in some patients, and does not have a favorable effect on β-thromboglobulin. β-thromboglobulin can activate platelets and can cause increased aggregation and has atherogenic potential. Therefore, lowering β-thromboglobulin is particularly advantageous. Patients with diabetes mellitus have an increased risk of thrombosis and accelerated atherogenesis and increased platelet adhesion and aggregation. Four general platelet regions or functional units can be involved in aberrant chemistry, structure and/or function. These include (1) the membrane, (2) granules, (3) intermediary metabolism, and (4) other factors and/or platelet responses to various substances. In regard to the abnormalities of the membrane, there is an increased binding of fibrinogen in diabetic rats and increased membrane rigidity. There are increases in glycoprotein Ib and glycoprotein IIb/IIIa. Related to granule function, increased levels of plasma serotonin, histamine and P-thromboglobulin are found. Alterations of intermediary metabolism involving the-prostaglandin pathways, arachidonic acid, Vitamin E, and lipids have also been reported. Other factors which are not well characterized include abnormalities of stem cell response to growth factors and thrombopoiesis, as noted indirectly through alterations of platelet volumes. It is believed that these platelet abnormalities result in increased thrombosis and/or an acceleration of the atherosclerotic process in at least some patients with diabetes mellitus.

The amount of pantethine added can range from about 50 mg to about 3000 mg per unit dose. Pantethine provides its therapeutic effect in whole or in part through acetyl CoA chemistry which is synergistic to (supplements, complements and enhances) the mechanisms of the multiple release combination of niacin/IHN or other analogue or derivative of nicotinic acid.

Using a multiple release composition as set forth herein allows for dosing of both nicotinic acid and IHN or other analogue or derivative of nicotinic acid in concentrations that will not only minimize flushing but will allow for the use of lower doses of both ingredients than are normally required to generate significant reduction in serum lipids while reducing the undesirable side effects of taking large doses of niacin.

Other ingredients known to have a beneficial effect on serum lipids and to lower cholesterol, such as, but not limited to policosanol, phytosterols, tocotrienols, calcium, statin drugs, fibrates, bile acid sequestrants, and guar gum can be added—to this combination of nicotinic acid and IHN or other analogue or derivative of nicotinic acid, with or without pantethine, to increase the cardiovascular benefits of the composition. If present, these ingredients can be added in a therapeutically effective quantity. In addition ingredients such as Llysine, L-proline, vitamin C, vitamin E, or other antioxidants that prevent lipid peroxidation can be added to the composition. If present, these ingredients can also be added in a therapeutically effective quantity.

Accordingly, another embodiment of the invention comprises a method of treating a dysregulation of lipid metabolism comprising the step of administering a quantity of a composition according to the present invention to a subject suffering from or at risk of suffering from a dysregulation of lipid metabolism in a quantity effective to treat the dysregulation of lipid metabolism. As used herein, the term “treat” refers to the causation of any detectable improvement of lipid metabolism that is clinically significant and does not require or demand a cure. The improvement of lipid metabolism can be evidenced by a detectable decrease in serum cholesterol, serum lipids, or serum triglycerides. The improvement of lipid metabolism can also be evidenced by a detectable objective or subjective status of the patient, including increased energy, decreased fatigue, decreased pain such as that of angina pectoris or intermittent claudication, or other clinical indicator detectable by the treating physician.

The subject can be a human or a socially or economically important animal such as, but not limited to, a dog, a cat, a horse, a cow, a sheep, a goat, or a pig. Methods according to the present invention are not limited to the treatment of humans.

The exact formulation, route of administration and dosage can be chosen by the individual physician in view of the patient's condition. (See e.g. Fingl et al., The Pharmacological Basis of Therapeutics, 1975, Ch. 1 p. 1). It should be noted that the attending physician would know how to and when to terminate, interrupt, or adjust administration due to toxicity or to organ dysfunctions. Conversely, the attending physician would also know to adjust treatment to higher levels if the clinical response were not adequate (precluding toxicity). The magnitude of an administered dose in the management of the disorder of interest will vary with the severity of the condition to be treated and to the route of administration. The severity of the condition may, for example, be evaluated, in part, by standard prognostic evaluation methods. Further, the dose and perhaps the dose frequency, will also vary according to the age, body weight, and response of the individual patient. A program comparable to that discussed above may be used in veterinary medicine.

Depending on the specific conditions being treated, such compositions may be formulated and administered systemically or locally. Typically, administration is systemic. Techniques for formulation and administration may be found in Remington's Pharmaceutical Sciences, 18th ed., Mack Publishing Co., Easton, Pa. (1990). Suitable routes may include oral, rectal, transdermal, vaginal, transmucosal, or intestinal administration; parenteral delivery, including intramuscular, subcutaneous, intramedullary injections, as well as intrathecal, direct intraventricular, intravenous, intraperitoneal, intranasal, or intraocular injections, just to name a few. Typically, oral administration is preferred.

Typically, compositions according to the present invention are administered once daily, in the afternoon, i.e., from about 4 p.m. to about 6 p.m., with a snack or meal. However, other patterns of administration can be used; either the time of administration can be varied or the frequency of administration can be varied, depending on the condition being treated, its severity, the effects of the course of treatment, and the existence of conditions such as liver or kidney conditions that affect the pharmacokinetics of the composition.

Methods according to the present invention can be used to treat hyperlipidemia, hyper cholesterolemia, and hypertriglyceridemia in patients in which one or more of these dysregulations of lipid metabolism occur. Additionally, methods according to the present invention can be used to lower levels of blood lipids, blood cholesterol, or blood triglycerides in patients who have already experienced an episode of cardiovascular disease such as myocardial infarction or stroke or who are considered to be at risk of occurrence of cardiovascular disease, even when the levels of blood lipids, blood cholesterol, or blood triglycerides are within normal limits. In particular, methods according to the present invention using compositions containing nicotinic acid, IHN, and pantethine are useful for the treatment of subjects who are diabetic or have insulin resistance or who have been diagnosed with cardiovascular disease.

The invention described herein is unique in that it provides: (1) release of niacin through different pathways and at different times from a single daily dosage; (2) reduced or less flushing compared to dosages of niacin alone necessary to obtain the same effect; (3) reduced risk of elevating liver enzymes; (4) a reduction in the therapeutic doses of niacin and IHN or other analogue or derivative of nicotinic acid necessary to obtain the desired result if delivered independently; (5) single afternoon dosing, as opposed to dosing of 3-4 times a day of immediate release niacin or IHN, or a dosage at bed time of the extended release niacin; (6) additional health benefits for peripheral artery disease which is common in people with coronary heart disease by the use of IHN or other analogue or derivative of nicotinic acid; and (7) little or no elevation of blood glucose levels.

In addition to a novel multiple release of niacin and IHN or other analogue or derivative of nicotinic acid, if pantethine is included it adds favorable effects on acetyl CoA levels and possible benefits of HMG CoA reductase chemistry for a synergistic result.

Compositions according to the present invention can also be used for the treatment of cardiovascular disease, namely reversal of accumulation of cardiovascular plaques. Accordingly, another aspect of the present invention is a method for the treatment of cardiovascular disease comprising the step of administering a pharmaceutical composition according to the present invention to a subject suffering from or at risk for cardiovascular disease in a quantity effective to reverse accumulation of cardiovascular plaques.

Some of the additional benefits of this combination multiple release system includes reduction of anxiety, depression, PMS, peripheral vascular diseases such as thrombotic risk, intermittent claudication, Raynaud's disease, hypertension, or dysmenorrhea, vascular insifficiency and restless leg syndrome and treatment of metabolic syndrome due to insulin resistance. The combination is also beneficial in reducing fibrinogen and increasing blood viscosity, reducing or alleviating migrane headaches and treating alcoholism and skin diseases such as pruritis and sceleroderma. A further unique aspect of the composition described therein is that atherosclerosis is reduced because LDL absorption on the arteries is reduced. LPa has been found to be a major factor in the formation of plaque in the arteries. One end of the LPa molecule adheres to the vessel wall and this then acts as an anchor to grab circulating LDL. However, niacin circulating in the blood stream appears to “blunt” the exposed end of the LPa extending into the blood stream preventing the LDL from adhering to the vessel wall and then reducing plaque buildup.

### ADVANTAGES OF THE INVENTION

The present invention provides compositions and methods that are particularly effective for the treatment of hyperlipidemia, hypercholesterolemia, and hypertriglyceridemia, as well as for the treatment of cardiovascular disease. These compositions and methods are effective and do not cause side effects that have prevented the use of niacin for these purposes such as flushing and adverse interactions with other drugs such as statins. These compositions and methods can also be used prophylactically to prevent the occurrence of cardiovascular diseases such as stroke and myocardial infarction. They are well tolerated and also provide additional benefits by treating peripheral vascular conditions and hypertension, as well as cardiovascular disease. Additionally, they are suitable for use by subjects who have normal levels of blood lipids, cholesterol, or triglycerides, but who are at risk of cardiovascular disease or who have already experienced an episode of cardiovascular disease such as myocardial infarction or stroke.

With respect to ranges of values, the invention encompasses each intervening value between the upper and lower limits of the range to at least a tenth of the lower limit's unit, unless the context clearly indicates otherwise. Moreover, the invention encompasses any other stated intervening values and ranges including either or both of the upper and lower limits of the range, unless specificallyy excluded from the stated range.

Unless defined otherwise, the meanings of all technical and scientific terms used herein are those commonly understood by one of ordinary skill in the art to which this invention belongs. One of ordinary skill in the art will also appreciate that any methods and materials similar or equivalent to those described herein can also be used to practice or test this invention.

The publications and patents discussed herein are provided solely for their disclosure prior to the filing date of the present application. Nothing herein is to be construed as an admission that the present invention is not entitled to antedate such publication by virtue of prior invention. Further the dates of publication provided may be different from the actual publication dates which may need to be independently confirmed.

All the publications cited are incorporated herein by reference in their entireties, including all published patents, patent applications, literature references, as well as those publications that have been incorporated in those published documents. However, to the extent that any publication incorporated herein by reference refers to information to be published, applicants do not admit that any such information published after the filing date of this application to be prior art.

As used in this specification and in the appended claims, the singular forms include the plural forms. For example the terms “a,” “an,” and “the” include plural references unless the content clearly dictates otherwise. Additionally, the term “at least” preceding a series of elements is to be understood as referring to every element in the series. The inventions illustratively described herein can suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the future shown and described or any portion thereof, and it is recognized that various modifications are possible within the scope of the invention claimed. Thus, it should be understood that although the present invention has been specifically disclosed by preferred embodiments and optional features, modification and variation of the inventions herein disclosed can be resorted by those skilled in the art, and that such modifications and variations are considered to be within the scope of the inventions disclosed herein. The inventions have been described broadly and generically herein. Each of the narrower species and subgeneric groupings falling within the scope of the generic disclosure also form part of these inventions. This includes the generic description of each invention with a proviso or negative limitation removing any subject matter from the genus, regardless of whether or not the excised materials specifically resided therein. In addition, where features or aspects of an invention are described in terms of the Markush group, those schooled in the art will recognize that the invention is also thereby described in terms of any individual member or subgroup of members of the Markush group. It is also to be understood that the above description is intended to be illustrative and not restrictive. Many embodiments will be apparent to those of in the art upon reviewing the above description. The scope of the invention should therefore, be determined not with reference to the above description, but should instead be determined with reference to the appended claims, along with the full scope of equivalents to which such claims are entitled. Those skilled in the art will recognize, or will be able to ascertain using no more than routine experimentation, many equivalents to the specific embodiments of the invention described. Such equivalents are intended to be encompassed by the following claims.

I claim:

1. A composition for treatment of a dysregulation of lipid metabolism comprising:
about 1.0%_{w} to about 50%_{w} of nicotinic acid and about 50%_{w} to about 99%_{w} of a derivative or analogue of nicotinic acid.

2. The composition of claim 1 comprises:
(a) a quantity of nicotinic acid sufficient to saturate liver-enzymes during a period from about 0.5 hours to about 2.5 hours after administration of the pharmaceutical composition but insufficient to trigger significant generation of nicotinuric acid;
(b) a quantity of the derivative or analogue of nicotinic acid that is subject to hydrolysis after ingestion such that no more than about 10% of the derivative or analogue is hydrolyzed by about 8 hours after ingestion and such that substantially all of the derivative or analogue is hydrolyzed by about 12 hours after ingestion; and
(c) at least one pharmaceutically acceptable carrier.

3. The composition of claim 2 wherein the derivative or analogue of nicotinic acid is inositol hexanicotinate (IHN), said inositol hexanicotinate comprising one or more inositol isomers.

4. The composition of claim 2 wherein the derivative or analogue of nicotinic acid is selected from the group consisting of:
(a) nicotinyl alcohol tartrate and derivatives thereof;
(b) D-glucitol hexanicotinate and derivatives thereof;
(c) aluminum nicotinate and derivatives thereof;
(d) pentaerythrityl tetranicotinate (niceritrol) and derivatives thereof;
(e) D,L-α-tocopheryl nicotinate and derivatives thereof;
(f) nicotinamide;
(g) nicotinamide N-oxide;
(h) 6-hydroxynicotinamide;
(i) nicotinamide adenine dinucleotide (NAD);
(j) N-methyl-2-pyridine-8-carboxamide;
(k) N-methylnicotinamide;
(l) N-ribosyl-2-pyridone-5-carboxamide;
(m) N-methyl-4-pyridone-5-carboxamide;
(n) 1,3,3,6-tetranicotinyl-D-fructose (Bradilan);
(o) an ester of ronicol (nicotinyl alcohol) selected from the group consisting of a lower alkyl ester, an aryl ester, an aralkyl ester, and an alkaryl ester; and
(p) an ester of nicotinic acid selected from the group consisting of a lower alkyl ester, an aryl ester, an aralkyl ester, and an alkaryl ester.

5. The composition of claim 2 wherein the analogue or derivative of nicotinic acid is selected from the group consisting of:
(a) an ester or amide of 3-pyridinecarboxylic acid;
(b) an ester or amide of 3-pyridinecarbonitrile-1-oxide;
(c) a monoester, diester, monoamide, or diamide of 3,4pyridinedicarboxylic acid;
(d) an ester or amide of nipecotic acid;
(e) an ester or amide of 1-methyl-3-piperidinecarboxylic acid;
(f) an esteror amide of 3-carboxymethylpyridine;
(g) an ester or amide of 3-carboxyethylpyridine;
(h) an ester or amide of 3-carboxy-1-methylpyridinium chloride;
(i) an ester or amide of 5-bromo-3-pyridinecarboxylic acid;
(j) an ester or amide of β-oxo-3-pyridinepropionic acid;
(k) an ester or amide of 2-acetyl-3-pyridinecarboxylic acid;
(l) a monoester, diester, monoamide, or diamide of [2,2′dipyridine]-5,5′-dicarboxylic acid;
(m) an ester or amide of 2-chloro-3-pyridinecarboxylic acid;
(n) an ester or amide of 6-chloro-3-pyridinecarboxylic acid; and
(o) an ester or amide of 6′hydroxy-pyridinecarboxylic acid.

6. The composition of claim 2 wherein the composition is formulated for-oral administration.

7. The composition of claim 2 wherein the composition comprises from about 1 mg to about 2000 mg nicotinic acid per unit dose.

8. The composition of claim 3 wherein the composition comprises from about 1 mg to about 2000 mg nicotinic acid per unit dose.

9. The composition of claim 2 wherein the composition comprises nicotinic acid in a quantity per unit dose selected from the group consisting of 1 mg per unit dose, 10 mg per unit dose, 15 mg per unit dose, 20 mg per unit dose, 25 mg per unit dose, 30 mg per unit dose, 35 mg per unit dose, 40 mg per unit dose, 50 mg per unit dose, 60 mg per unit dose, 70 mg per unit dose, 80 mg per unit dose, 90 mg per unit dose, 100 mg per unit dose, 150 mg per unit dose, 200 mg per unit dose, 250 mg per unit dose, 300 mg per unit dose, 350 mg per unit dose, 400 mg per unit dose, 450 mg per unit dose, 500 mg per unit dose, 600 mg per unit dose, 700 mg per unit dose, 800 mg per unit dose, 900 mg per unit dose, 1000 mg per unit dose, 1100 mg per unit dose, 1200 mg per unit dose, 1300 mg per unit dose, 1400 mg per unit dose, 1500 mg per unit dose, 1600 mg per unit dose, 1700 mg per unit dose, 1800 mg per unit dose, 1900 mg per unit dose, and 2000 mg per unit dose.

10. The composition of claim 3 wherein the composition comprises nicotinic acid in a quantity per unit dose selected from the group consisting of 1 mg per unit dose, 10 mg per unit dose, 15 mg per unit dose, 20 mg per unit dose, 25 mg per unit dose, 30 mg per unit dose, 35 mg per unit dose, 40 mg per unit dose, 50 mg per unit dose, 60 mg per unit dose, 70 mg per unit dose, 80 mg per unit dose, 90 mg per unit dose, 100 mg per unit dose, 150 mg per unit dose, 200 mg per unit dose, 250 mg per unit dose, 300 mg per unit dose, 350 mg per unit dose, 400′ mg per unit dose, 450 mg per unit dose, 500 mg per unit dose, 600 mg per unit dose, 700 mg per unit dose, 800 mg per unit dose, 900 mg per unit dose, 1000 mg per unit dose, 1100 mg per unit dose, 1200 mg per unit dose, 1300 mg per unit dose, 1400 mg per unit dose, 1500 mg per unit dose, 1600 mg per unit dose, 1700 mg per unit dose, 1800 mg per unit dose, 1900 mg per unit dose, and 2000 mg per unit dose.

11. The composition of claim 8 wherein the composition comprises from about 100 mg to about 3000 mg of IHN per unit dose.

12. The composition of claim 1 further comprising pantethine or phosphatidylinositol or pantethine and phosphatidylinositol in therapeutically effective quantities.

13. The composition of claim 12 wherein the composition comprises from about 50 mg to about 3000 mg of pantethine per unit dose and from about 250 mg to about 6 gm of phosphatidylinositol.

14. The composition of claim 3 further comprising pantethine or phosphatidylinositol or pantithine and phosphatidylinositol in therapeutically effective quantities.

15. The composition of claim 14 wherein the composition comprises from about 50 mg to about 3000 mg of pantethine per unit dose and from about 250 mg to about 6 gm of phosphatidylinositol.

16. The composition of claim 11 further comprising pantethine or phosphatidylinositol or pantithine and phosphatidylinositol in therapeutically effective quantities.

17. The composition of claim 16 wherein the composition comprises from about 50 mg to about 3000 mg of pantethine per unit dose and from about 250 mg to about 6 gm of phosphatidylinositol.

18. The composition of claim 1 further comprising at least one ingredient selected from the group consisting of policosanol, phytosterols, tocotrienols, calcium, statin drugs, fibrates, bile acid sequestrants, and guar gum in a therapeutically effective quantity.

19. The composition of claim 3 further comprising at least one ingredient selected from the group consisting of policosanol, phytosterols, tocotrienols, calcium, statin drugs, fibrates, bile acid sequestrants, and guar gum in a therapeutically effective quantity.

20. The composition of claim 12 further comprising at least one ingredient selected from the group consisting of policosanol, phytosterols, tocotrienols, calcium, statin drugs, fibrates, bile acid sequestrants, and guar gum in a therapeutically effective quantity.

21. The composition of claim 14 further comprising at least one ingredient selected from the group consisting of policosanol, phytosterols, tocotrienols, calcium, statin drugs, fibrates, bile acid sequestrants, and guar gum in a therapeutically effective quantity.

22. The composition of claim 1 further comprising at least one ingredient selected from the group consisting of L-lysine, L-proline, vitamin C, vitamin E, and another antioxidant effective in preventing lipid peroxidation in a therapeutically effective quantity.

23. The composition of claim 3 further comprising at least one ingredient selected from the group consisting of L-lysine, L-proline, vitamin C, vitamin E, and another antioxidant effective in preventing lipid peroxidation in a therapeutically effective quantity.

24. The composition of claim 12 further comprising at least one ingredient selected from the group consisting of L-lysine, L-proline, vitamin C, vitamin E, and another antioxidant effective in preventing lipid peroxidation in a therapeutically effective quantity.

25. The composition of claim 14 further comprising at least one ingredient selected from the group consisting of L-lysine, L-proline, vitamin C, Vitamin E, and another antioxidant effective in preventing lipid peroxidation in a therapeutically effective quantity.

26. The composition of claim 1 further comprising a matrix that slows the disintegration of the composition, in order that the level of the nicotinic acid in the blood due to the quantity of nicotinic acid sufficient to saturate liver enzymes in the composition is maximized at about 2 to 3 hours after ingestion instead of at about 45 minutes without the matrix.

27. The composition of claim 26 wherein the composition is in the form of a tablet.

28. The composition of claim 3 further comprising a matrix that slows the disintegration of the composition, in order that the level of the nicotinic acid in the blood due to the quantity of nicotinic acid sufficient to saturate liver enzymes in the composition is maximized at about 2 to 3 hours after ingestion instead of at about 45 minutes without the matrix.

29. The composition of claim 28 wherein the composition is in the form of a tablet.

30. The composition of claim 12 further comprising a matrix that slows the disintegration of the composition, in order that the level of the nicotinic acid in the blood due to the quantity of nicotinic acid sufficient to saturate liver enzymes in the composition is maximized at about 2 to 3 hours after ingestion instead of at about 45 minutes without the matrix.

31. The composition of claim 30 wherein the composition is in the form of a tablet.

32. The composition of claim 14 further comprising a matrix that slows the disintegration of the composition, in order that the level of the nicotinic acid in the blood due to the quantity of nicotinic acid sufficient to saturate liver enzymes in the composition is maximized at about 2 to 3 hours after ingestion instead of at about 45 minutes without the matrix.

33. The composition of claim 32 wherein the composition is in the form of a tablet.

34. A method of treating a dysregulation of lipid metabolism comprising the step of administering the composition of claim 2 to a subject suffering from or at risk of suffering from a dysregulation of lipid metabolism in a quantity effective to treat the dysregulation of lipid metabolism.

35. The method of claim 34 wherein the derivative or analogue of nicotinic acid that is subject to hydrolysis is an ester or amide of nicotinic acid.

36. The method of claim 35 wherein the ester or amide of nicotinic acid is inositol hexanicotinate (IHN), said inositol hexanicotinate comprising one or more isomers of inositol.

37. The method of claim 35 wherein the ester or amide of nicotinic acid is selected from the group consisting of:
(a) nicotinyl alcohol tartrate and derivatives thereof;
(b) D-glucitol hexanicotinate and derivatives thereof;
(c) aluminum nicotinate and derivatives thereof;
(d) pentaerythrityl tetranicotinate (niceritrol) and derivatives
(e) D,L-α-tocopheryl nicotinate and derivatives thereof;
(f) nicotinamide;
(g) nicotinamide N-oxide;
(h) 6-hydroxynicotinamide;
(i) nicotinamide adenine dinucleotide (NAD);
(j) N-methyl-2-pyridine-8-carboxamide;
(k) N-methylnicotinamide;
(l) N-ribosyl-2-pyridone-5-carboxamide;
(m) N-methyl-4-pyridone-5-carboxamide;
(n) 1;3,3,6-tetranicotinyl-D-fructose (Bradilan);
(o) an ester of ronicol (nicotinyl alcohol) selected from the group consisting of a lower alkyl ester, an aryl ester, an aralkyl ester, and an alkaryl ester; and
(p) an ester of nicotinic acid selected from the group consisting of a lower alkyl ester, an aryl ester, an aralkyl ester, and an alkaryl ester.

38. The method of claim 34 wherein the analogue or derivative of nicotinic acid is selected from the group consisting of:
(a) an ester or amide of 3-pyridinecarboxylic acid;
(b) an ester or amide of 3-pyridinecarbonitrile-1-oxide;
(c) a monoester, diester, monoamide, or diamide of 3,4 pyridinedicarboxylic acid;
(d) an ester or amide of nipecotic acid;
(e) an ester or amide of 1-methyl-3-piperidinecarboxylic acid;
(f) an ester or amide of 3-carboxymethylpyridine;
(g) an ester or amide of 3-carboxyethylpyridine;
(h) an ester or amide of 3-carboxy-1-methylpyridinium chloride;
(i) an ester or amide of 5-bromo-3-pyridinecarboxylic acid;
(j) an ester or amide of β-oxo-3-pyridinepropionic acid;
(k) an ester or amide of 2-acetyl-3-pyridinecarboxylic acid;
(l) a monoester, diester, monoamide, or diamide of [2,2′dipyridine]-5,5′-dicarboxylic acid;
(m) an ester or amide of 2-chloro-3-pyridinecarboxylic acid;
(n) an ester or amide of 6-chloro-3-pyridinecarboxylic acid; and
(o) an ester or amide of 6-hydroxy-pyridinecarboxylic acid.

39. The method of claim 34 wherein the composition is formulated for oral administration and administered orally.

40. The method of claim 34 wherein the composition comprises from about 1 mg to about 2000 mg nicotinic acid per unit dose.

41. The method of claim 36 wherein the composition comprises from about 1 mg to about 2000 mg nicotinic acid per unit dose.

42. The method of claim 40 wherein the composition comprises nicotinic acid in a quantity per unit dose selected from the group consisting of 1 mg per unit dose, 10 mg per unit dose, 15 mg per unit dose, 20 mg per unit dose, 25 mg per unit dose, 30 mg per unit dose, 35 mg per unit dose, 40 mg per unit dose, 50 mg per unit dose, 60 mg per unit dose, 70 mg per unit dose, 80 mg per unit dose, 90 mg per unit dose, 100 mg per unit dose, 150 mg per unit dose, 200 mg per unit dose, 250 mg per unit dose, 300 mg per unit dose, 350 mg per unit dose, 400 mg per unit dose, 450 mg per unit dose, 500 mg per unit dose, 600 mg per unit dose, 700 mg per unit dose, 800 mg per unit dose, 900 mg per unit dose, 1000 mg per unit dose, 1100 mg per unit dose, 1200 mg per unit dose, 1300 mg per unit dose, 1400 mg per unit dose, 1500 mg per unit dose, 1600 mg per unit dose, 1700 mg per unit dose, 1800 mg per unit dose, 1900 mg per unit dose, and 2000 mg per unit dose.

43. The method of claim 41 wherein the composition comprises nicotinic acid in a quantity per unit dose selected from the group consisting of 1 mg per unit dose, 10 mg per unit dose, 15 mg per unit dose, 20 mg per unit dose, 25 mg per unit dose, 30 mg per unit dose, 35 mg per unit dose, 40 mg per unit dose, 50 mg per unit dose, 60 mg per unit dose, 70 mg per unit dose, 80 mg per unit dose, 90 mg per unit dose, 100 mg per unit dose, 150 mg per unit dose, 200 mg per unit dose, 250 mg per unit dose, 300 mg per unit dose, 350 mg per unit dose, 400 mg per unit dose, 450 mg per unit dose, 500 mg per unit dose, 600 mg per unit dose, 700 mg per unit dose, 800 mg per unit dose, 900 mg per unit dose, 1000 mg per unit dose, 1100 mg per unit dose, 1200 mg per unit dose, 1300 mg per unit dose, 1400 mg per unit dose, 1500 mg per unit dose, 1600 mg per unit dose, 1700 mg per unit dose, 1800 mg per unit dose, 1900 mg per unit dose, and 2000 mg per unit dose.

44. The method of claim 41 wherein the composition comprises from about 100 mg to about 3000 mg of IHN per unit dose.

45. The method of claim 34 Wherein the composition further comprises pantethine in a therapeutically effective quantity.

46. The method of claim 45 wherein the composition comprises from about 50 mg to about 3000 mg of pantethine per unit dose.

47. The method of claim 36 wherein the composition further comprises pantethine in a therapeutically effective quantity.

48. The method of claim 47 wherein the composition comprises from about 50 mg to about 3000 mg of pantethine per unit dose.

49. The method of claim 45 wherein the composition further comprises pantethine in a therapeutically effective quantity.

50. The method of claim 49 wherein the composition comprises from about 50 mg to about 3000 mg of pantethine per unit dose.

51. The method of claim 34 wherein the composition further comprises at least one ingredient selected from the group consisting of policosanol, phytosterols, tocotrienols, calcium, statin drugs, fibrates, bile acid sequestrants, and guar gum in a therapeutically effective quantity.

52. The method of claim 36 wherein the composition further comprises at least one ingredient selected from the group consisting of policosanol, phytosterols, tocotrienols, calcium, statin drugs, fibrates, bile acid sequestrants, and guar gum in a therapeutically effective quantity.

53. The method of claim 45 wherein the composition further comprises at least one ingredient selected from the group consisting of policosanol, phytosterols, tocotrienols, calcium, statin drugs, fibrates, bile acid sequestrants, and guar gum in a therapeutically effective quantity.

54. The method of claim 47 wherein the composition further comprises at least one ingredient selected from the group consisting of policosanol, phytosterols, tocotrienols, calcium, statin drugs, fibrates, bile acid sequestrants, and guar gum in a therapeutically effective quantity.

55. The method of claim 34 wherein the composition further comprises at least one ingredient selected from the group consisting of L-lysine, L-proline, vitamin C, vitamin E, and another antioxidant effective in preventing lipid peroxidation in a therapeutically effective quantity.

56. The method of claim 36 wherein the composition further comprises at least one ingredient selected from the group consisting of L-lysine, L-proline, vitamin C, vitamin E, and another antioxidant effective in preventing lipid peroxidation in a therapeutically effective quantity.

57. The method of claim 45 wherein the composition further comprises at least one ingredient selected from the group consisting of L-lysine, L-proline, vitamin C, vitamin E, and another antioxidant effective in preventing lipid peroxidation in a therapeutically effective quantity.

58. The method of claim 47 wherein the composition further comprises at least one ingredient selected from the group consisting of L-lysine, L-proline, vitamin C, vitamin E, and another antioxidant effective in preventing lipid peroxidation in a therapeutically effective quantity.

59. The method of claim 34 wherein the composition further comprises a matrix that slows the disintegration of the composition, in order that the level of the nicotinic acid in the blood due to the quantity of nicotinic acid sufficient to saturate liver enzymes in the composition is maximized at about 2 to 3 hours after ingestion instead of at about 45 minutes without the matrix.

60. The method of claim 59 wherein the composition is in the form of a tablet.

61. The method of claim 34 wherein the dysregulation of lipid metabolism is hyperlipidemia.

62. The method of claim 34 wherein the dysregulation of lipid metabolism is hypercholesterolemia.

63. The method of claim 34 wherein the dysregulation of lipid metabolism is hypertriglyceridemia.

64. The method of claim 34 wherein the subject has experienced an episode of cardiovascular disease prior to administration of the composition.

65. The method of claim 34 wherein the subject is at risk of an episode of cardiovascular disease.

66. The method of claim 34 wherein the composition is administered once daily.

67. The method of claim 66 wherein the composition is administered in the afternoon.

68. The method of claim 47 wherein the subject is diabetic or has insulin resistance.

69. A method for the treatment of cardiovascular disease comprising the step of administering the composition of claim 2 to a subject suffering from or at risk for cardiovascular disease in a quantity effective to reverse accumulation of cardiovascular plaques.

70. The method of claim 69 wherein the derivative or analogue of nicotinic acid that is subject to hydrolysis is an ester or amide of nicotinic acid.

71. The method of claim 70 wherein the ester or amide of nicotinic acid is inositol hexanicotinate (IHN), and said inositol hexanicotinate comprises one or more isomers of inositol.

72. The method of claim 70 wherein the ester or amide of nicotinic acid is selected from the group consisting of:
(a) nicotinyl alcohol tartrate and derivatives thereof;
(b) D-glucitol hexanicotinate and derivatives thereof;
(c) aluminum nicotinate and derivatives thereof;
(d) pentaerythrityl tetranicotinate (niceritrol) and derivatives
(e) D,L-α-tocopheryl nicotinate and derivatives thereof;
(f) nicotinamide;
(g) nicotinamide N-oxide;
(h) 6-hydroxynicotinamide;
(i) nicotinamide adenine dinucleotide (NAD);
(j) N-methyl-2-pyridine-8-carboxamide;
(k) N-methylnicotinamide;
(1) N-ribosyl-2-pyridone-5-carboxamide;
(m) N-methyl-4-pyridone-5-carboxamide;
(n) 1,3,3,6-tetranicotinyl-D-fructose (Bradilan);
(o) an ester of ronicol (nicotinyl alcohol) selected from the group consisting of a lower alkyl ester, an aryl ester, an aralkyl ester, and an alkaryl ester; and
(p) an ester of nicotinic acid selected from the group consisting of a lower alkyl ester, an aryl ester, an aralkyl ester, and an alkaryl ester.

73. The method of claim 69 wherein the analogue or derivative of nicotinic acid is selected from the group consisting of:
(a) an ester or amide of 3-pyridinecarboxylic acid;
(b) an ester or amide of 3-pyridinecarbonitrile-1-oxide;
(c) a monoester, diester, monoamide, or diamide of 3,4-pyridinedicarboxylic acid;
(d) an ester or amide of nipecotic acid;
(e) an ester or amide of 1-methyl-3-piperidinecarboxylic acid;
(f) an ester or amide of 3-carboxymethylpyridine;
(g) an ester or amide of 3-carboxyethylpyridine;
(h) an ester or amide of 3-carboxy-1-methylpyridinium chloride;
(i) an ester or amide of 5-bromo-3-pyridinecarboxylic acid;
(j) an ester or amide of a-oxo-3-pyridinepropionic acid;
(k) an ester or amide of 2-acetyl-3-pyridinecarboxylic acid;
(l) a monoester, diester, monoamide, or diamide of [2,2′-dipyridine]-5,5′-dicarboxylic acid;
(m) an ester or amide of 2-chloro-3-pyridinecarboxylic acid;
(n) an ester or amide of 6-chloro-3-pyridinecarboxylic acid; and
(o) an ester or amide of 6-hydroxy-pyridinecarboxylic acid.

74. The method of claim 69 wherein the composition is formulated for oral administration and administered orally.

75. The method of claim 69 wherein the composition comprises from about 1 mg to about 2000 mg nicotinic acid per unit dose.

76. The method of claim 71 wherein the composition comprises from about 1 mg to about 2000 mg nicotinic acid per unit dose.

77. The method of claim 75 wherein the composition comprises nicotinic acid in a quantity per unit dose selected from the group consisting of 1 mg per unit dose, 10 mg per unit dose, 15 mg per unit dose, 20 mg per unit dose, 25 mg per unit dose, 30 mg per unit dose, 35 mg per unit dose, 40 mg per unit dose, 50 mg per unit dose, 60 mg per unit dose, 70 mg per unit dose, 80 mg per unit dose, 90 mg per unit dose, 100 mg per unit dose, 150 mg per unit dose, 200 mg per unit dose, 250 mg per unit dose, 300 mg per unit dose, 350 mg per unit dose, 400 mg per unit dose, 450 mg per unit dose, 500 mg per unit dose, 600 mg per unit dose, 700 mg per unit dose, 800 mg per unit dose, 900 mg per unit dose, 1000 mg per unit dose, 1100 mg per unit dose, 1200 mg per unit dose, 1300 mg per unit dose, 1400 mg per unit dose, 1500 mg per unit dose, 1600 mg per unit dose, 1700 mg per unit dose, 1800 mg per unit dose, 1900 mg per unit dose, and 2000 mg per unit dose.

78. The method of claim 76 wherein the composition comprises nicotinic acid in a quantity per unit dose selected from,the group consisting of 1 mg per unit dose, 10 mg per unit dose, 15 mg per unit dose, 20 mg per unit dose, 25 mg per unit dose, 30 mg per unit dose, 35 mg per unit dose, 40 mg per unit dose, 50 mg per unit dose, 60 mg per unit dose, 70 mg per unit dose, 80 mg per unit dose, 90 mg per unit dose, 100 mg per unit dose, 150 mg per unit dose, 200 mg per unit dose, 250 mg per unit dose, 300 mg per unit dose, 350 mg per unit dose, 400 mg per unit dose, 450 mg per unit dose, 500 mg per unit dose, 600 mg per unit dose, 700 mg per unit dose, 800 mg per unit dose, 900 mg per unit dose, 1000 mg per unit dose, 1100 mg per unit dose, 1200 mg per unit dose, 1300 mg per unit dose, 1400 mg per unit dose, 1500 mg per unit dose, 1600 mg per unit dose, 1700 mg per unit dose, 1800 mg per unit dose, 1900 mg per unit dose, and 2000 mg per unit dose.

79. The method of claim 76 wherein the composition comprises from about 100 mg to about 5 gm of IHN per unit dose.

80. The method of claim 69 wherein the composition further comprises pantethine in a therapeutically effective quantity.

81. The method of claim 80 wherein the composition comprises from about 50 mg to about 3000 mg of pantethine per unit dose.

82. The method of claim 71 wherein the composition further comprises pantethine in a therapeutically effective quantity.

83. The method of claim 82 wherein the composition comprises from about 50 mg to about 3000 mg of pantethine per unit dose.

84. The method of claim 75 wherein the composition further comprises pantethine in a therapeutically effective quantity.

85. The method of claim 84 wherein the composition comprises from about 50 mg to about 3000 mg of pantethine per unit dose.

86. The method of claim 69 wherein the composition further comprises at least one ingredient selected from the group consisting of policosanol, phytosterols, tocotrienols, calcium, statin drugs, fibrates, bile acid sequestrants, and guar gum in a therapeutically effective quantity.

87. The method of claim 71 wherein the composition further comprises at least one ingredient selected from the group consisting of policosanol, phytosterols, tocotrienols, calcium, statin drugs, fibrates, bile acid sequestrants, and guar gum in a therapeutically effective quantity.

88. The method of claim 75 wherein the composition further comprises at least one ingredient selected from the group consisting of policosanol, phytosterols, tocotrienols, calcium, statin drugs, fibrates, bile acid sequestrants, and guar gum in a therapeutically effective quantity.

89. The method of claim 82 wherein the composition further comprises at least one ingredient selected from the group consisting of policosanol, phytosterols, tocotrienols, calcium, statin drugs, fibrates, bile acid sequestrants, and guar gum in a therapeutically effective quantity.

90. The method of claim 69 wherein the composition further comprises at least one ingredient selected from the group consisting of L-lysine, L-proline, Vitamin C, vitamin E, and another antioxidant effective in preventing lipid peroxidation in a therapeutically effective quantity.

91. The method of claim 71 wherein the composition further comprises at least one ingredient selected from the group consisting of L-lysine, L-proline, vitamin C, vitamin E, and another antioxidant effective in preventing lipid peroxidation in a therapeutically effective quantity.

92. The method of claim 75 wherein the composition further comprises at least one ingredient selected from the group consisting of L-lysine, L-proline, vitamin C, vitamin E, and another antioxidant effective in preventing lipid peroxidation in a therapeutically effective quantity.

93. The method of claim 82 wherein the composition further comprises at least one ingredient selected from the group consisting of L-lysine, L-proline, vitamin C, vitamin E, and another antioxidant effective in preventing lipid peroxidation in a therapeutically effective quantity.

94. The method of claim 69 wherein the composition further comprises a matrix that slows the disintegration of the composition, in order that the level of the nicotinic acid in the blood due to the quantity of nicotinic acid sufficient to saturate liver enzymes in the composition is maximized at about 2 to 3 hours after ingestion instead of at about 45 minutes without the matrix.

95. The method of claim 94 wherein the composition is in the form of a tablet.

96. The composition of claim 1 wherein the derivative or analogue of nicotinic acid is inositol hexaniacinate, said inositol hexaniacinate comprising one or more inositol isomers.
